(19) United States
(12) Patent Application Publication
(10) Pub. No.: US 2025/0121114 A1
(43) Pub. Date: Apr. 17, 2025

(54) MACROPOROUS HYDROXYAPATITE COMPOSITION AND METHODS OF MAKING SUCH

(71) Applicant: CAVIX AB, Uppsala (SE)

(72) Inventors: Michael Wayne Trevino PUJARI-PALMER,, Uppsala (SE); Håkan ENGQVIST, Uppsala (SE)

(21) Appl. No.: 18/834,249

(22) PCT Filed: Feb. 15, 2023
(86) PCT No.: PCT/SE2023/050132

§ 371 Date: Jul. 30, 2024

(30) Foreign Application Priority Data
Feb. 16, 2022 (SE) 2250154-8

(51) Int. Cl. A61L 27/12; A61L 27/56; A61L 27/58; C04B 12/02; C04B 28/34; C04B 111/00

(52) U.S. Cl. CPC A61L 27/12; A61L 27/56; A61L 27/58; C04B 12/02; C04B 28/344; A61L 2430/02; C04B 2111/00836

(57) ABSTRACT

This invention relates to a macroporous cement composition suitable for use as a bone void filler. The macroporous cement composition comprises hydroxyapatite and β-calcium pyrophosphate. The porosity of the composition is 60-80 vol %. The invention also relates to methods of manufacturing such a macroporous cement composition and a putty formulation comprising such a macroporous cement composition.

### FIELD OF THE INVENTION

The present invention relates to a macroporous cement composition, in particular to a macroporous hydroxyapatite composition and methods of making such.

### BACKGROUND OF THE INVENTION

The interest in bone implants, such as bone void fillings have been a hot topic for researchers during several years. Calcium phosphate-based cement (CPC) material are considered ideal for bone replacement since they resemble the mineral phase of bone. Calcium phosphate-based cement materials are both biocompatible and biodegradable, i.e., degrade with time and is replaced with new healthy tissue, both of which are important properties for bone implants.

The aim of a bone void filling material is to have a fast resorption rate, mirroring an equally fast formation of new bone. The bone void filling material should ideally work as a template for new bone formation and prevent the formation of fibrotic tissue within the bone void. The presence of pores in a bone void filling material help to increase the bone ingrowth, which decreases the risk for implant failure.

WO 2015/162597 discloses a method for making a porous, chemically bonded ceramic shaped article. A porous, chemically bonded ceramic shaped article having interconnected pores, a total porosity of at least about 50%, and a macroporosity of at least about 30% can be formed by such methods.

‘Fabrication of microporous cement scaffolds using PEG particles: In vitro evaluation with induced pluripotent stem cell-derived mesenchymal progenitors’ by M. Sladkova et al, Materials Science and Engineering 69 (2016) 640-652 discloses macroporous calcium phosphate cements (CPS) and a fabrication method of making such. The method enables rapid, inexpensive and reproducible construction of macroporous CPC scaffolds with tunable architecture for potential use in dental and orthopedic applications.

The prior art represents advantages, however there is still a need for an improved porous cement composition having beneficial degradation rates for bone formation.

### SUMMARY OF THE INVENTION

The object of the present invention is to provide a cement composition suitable for use as a bone void filler.

This is achieved by the macroporous cement composition and method as defined in the independent claims.

An aspect of the invention relates to a macroporous cement composition comprising 80-95 wt % hydroxyapatite and β-calcium pyrophosphate. The porosity of the macroporous cement composition is 60-80 vol % as determined using Archimedes method.

In one embodiment of the invention, the macroporous cement composition further comprises α-tricalcium phosphate.

In one embodiment of the invention, the macroporous cement composition comprises 80-95 wt % hydroxyapatite, 0.1-10 wt % β-calcium pyrophosphate, preferably 1-10 wt % β-calcium pyrophosphate, and <10 wt % α-tricalcium phosphate.

In one embodiment of the invention, the macroporous cement composition comprises around 90 wt % hydroxyapatite, 0.1-10 wt % β-calcium pyrophosphate, preferably 1-10 wt % β-calcium pyrophosphate, and <10 wt % α-tricalcium phosphate.

In one embodiment of the invention, 25-75% of the total pore volume as determined by Archimedes method is constituted by pores having a pore diameter of 100-600 μm as determined by micro-computed tomography.

In one embodiment of the invention, the mean, or average, particle size of the β-calcium pyrophosphate in the macroporous cement composition is 500 nm-10 μm as determined by laser diffraction.

In one embodiment of the invention, the average particle size of the cement composition is 50-800 μm, preferably 50-600 μm as determined by sieving and/or scanning electron microscopy (SEM).

Another aspect of the invention relates to a method of manufacture a macroporous cement composition, wherein the method comprises the steps of:
mixing α-tricalcium phosphate, β-calcium pyrophosphate, and a sacrificial phase forming a dry powder mix, wherein all components are in solid form;
mixing the dry powder mix and a liquid forming a paste;
curing the paste at a temperature above room temperature, preferably at 50-60° C. for 20-30 hours;
terminating the chemical reaction associated with the curing after a predetermined time period selected so that a predetermined amount of α-tricalcium phosphate remains unreacted forming a solid cement composition, the predetermined amount being above 1 wt %, preferably above 3 wt %;
leaching the sacrificial phase from the solid cement composition; and
drying at a temperature above room temperature, preferably at 50-60° C. for 20-30 hours, to form the macroporous cement composition.

In one embodiment of the invention, the average particle size of the β-calcium pyrophosphate particles is 500 nm-10 μm as determined by sieving and/or SEM.

In one embodiment of the invention, the sacrificial phase is polyethylene glycol.

In one embodiment of the invention, the liquid is water and mixing the dry powder mixture comprises mixing the dry powder mixture with 0.4-0.6 mL water/g of the dry powder mix.

In one embodiment of the invention, terminating the chemical reaction comprises submerging the composition formed in the curing step in a solvent.

A further aspect of the invention relates to a putty formulation comprising the macroporous cement composition according to the invention.

The scope of the invention is defined by the claims. Any references in the description to methods of treatment refer to compounds, pharmaceutical compositions and medicaments of the present invention for use in a method for treatment of a human or animal body by therapy (or for diagnosis).

In the following, the invention will be described in more detail, by way of example only, with regard to non-limiting embodiments thereof, reference being made to the accompanying drawings.

### BRIEF DESCRIPTION OF THE DRAWINGS

FIG. 1 a) is a scanning electron microscope (SEM) image showing the macroporous cement composition in the form of granules according to the invention, b) is a graph showing the porosity at the y-axis against the amount of hydroxyapatite (HA) at the x-axis for four examples of the macroporous cement composition according to the invention;

FIG. 2 is a photograph showing the macroporous cement composition according to the invention;

FIG. 3 is a flow-chart of a method according to the invention;

FIG. 4 is a graph showing the amount of HA at the y-axis against the amount of coarse β-calcium pyrophosphate (β-CPP) at the x-axis for six examples of the macroporous cement composition according to the invention and two comparative examples;

FIG. 5 a) and b) are SEM images showing the macroporous cement composition according to examples of the invention;

FIG. 6 is a set of micro-computed tomography (μ-CT) images showing the macroporous cement composition according to examples of the invention; and

FIG. 7 is a graph showing the pore size distribution for 10 samples as the pore size (μm) on the x-axis vs. the fraction or % of total porosity at the y-axis.

### DEFINITIONS AND ABBREVIATIONS

Bioactive—able to stimulate cells and/or form bonds between the tissue and the bioactive phase;
Biocompatible—able to be in contact with a living system, e.g., cells, tissues, without producing adverse effects;
Macropores—pores with a diameter >100 μm;
Vol %—abbreviation for volume percent, i.e., percentage of total volume;
Wt %—abbreviation for weight percent, i.e., percentage of total weight;
HA—abbreviation for hydroxyapatite, chemical formula Ca₅(PO₄)₃(OH) or Ca₁₀(PO₈)₆(OH)₂;
β-CCP—abbreviation for β-calcium pyrophosphate, chemical formula Ca₂P₂O₇;
α-TCP—abbreviation for α-tricalcium phosphate, chemical formula Ca₃(PO₄)₂;
OCP—abbreviation for octacalcium phosphate, chemical formula Ca₈H₂(PO₄)₆·5H₂O; and
DCP—abbreviation for dicalcium phosphate, chemical formula Ca₂HPO₄ or Ca₂HPO₄·2H₂O.

### DETAILED DESCRIPTION

Adult humans have 206 different bones in their body. Bone is continuously remodeled during a person's lifetime, old and malfunctional bone is degraded and replaced with new bone. The cells present in bone are osteoclasts, osteoblasts, and osteocytes. They are responsible for the degradation and remodeling of bone. Ideally, a bone void filler material should function as a template for new bone formation rather than being a permanent bone substitute. Two main mechanisms are responsible for bone ingrowth into bone void fillers:
Osteoclastic degradation, i.e., that the bone void fillers are degraded in a similar way as natural bone and then replaced with new bone; and
Resorption through dissolution of the bone void filler material.

A faster bone ingrowth and/or a higher resorption rate can be achieved by the incorporation of macropores in the bone void filler material, i.e., pores with a diameter >100 μm. In addition, the composition of the bone void filler influences the bone ingrowth as well.

Typically, a bone void filler comprises one or more calcium phosphate phases. Examples of calcium phosphate phases include hydroxyapatite (HA, Ca₅(PO₄)₃(OH) or more commonly Ca₁₀(PO₈)₆(OH)₂), β-calcium pyrophosphate (β-CPP, Ca₂P₂O₇), α-tricalcium phosphate (α-TCP, Ca₃(PO₄)₂), octacalcium phosphate (OCP, Ca₈H₂(PO₄)₆·5H₂O), and dicalcium phosphate (DCP, Ca₂HPO₄ or Ca₂HPO₄·2H₂O). Different calcium phosphate phases have different properties, both in vitro and in vivo. Basically, all calcium phosphate phases are biocompatible and most of them are also bioactive. However, they have different dissolution/degradation rates in vivo (and in vitro). As mentioned above, this influences the rate of bone ingrowth and hence the effect of the bone void filler.

Hydroxyapatite (HA) is a form of calcium apatite with the formula Ca₅(PO₄)₃(OH) or Ca₁₀(PO₄)₆(OH)₂. Around 50 vol % of the human bone tissue is composed of hydroxyapatite. It is a widely studied material and suitable for use as a bone void filler or bone implant. Hydroxyapatite is known to be biocompatible and it is moderately bioactive. A bioactive calcium phosphate phase has the ability to stimulate cells and/or form bonds between the bone tissue and the bioactive phase, which is beneficial for a bone void filler. Hydroxyapatite does not degrade rapidly or release bioactive ions as rapidly as other, more soluble, calcium phosphates.

B-Calcium pyrophosphate is a bioactive phase, or bioactive material, that can react with the bone cells, or bone tissue. B-Calcium pyrophosphate is an insoluble calcium salt with the chemical formula Ca₂P₂O₇, it can be anhydrous or hydrous. Even if it is insoluble in vitro it is degraded quite rapidly in vivo where it can promote cell adhesion and tissue formation. It has been widely studied for use as a bone tissue repair material.

An aspect of the invention relates to a macroporous cement composition comprising 80-95 wt % hydroxyapatite, Ca₁₀(PO₄)₆(OH)₂, wherein the balance comprises β-calcium pyrophosphate, Ca₂P₂O₇, and wherein the porosity of the macroporous cement composition is 60-80 vol % as determined using Archimedes method.

Hence, the macroporous cement composition comprises 80-95 wt % hydroxyapatite and β-calcium pyrophosphate, and has a porosity of 60-80 vol % as determined using Archimedes method.

The term ‘porosity’ means total porosity, i.e., all volume in the macroporous cement composition that is empty space or voids, i.e., the total volume of pores that are equal to or below 600 μm in diameter. The porosity is given in vol % and calculated using helium (He) pycnometry or Archimedes method. Both He pycnometry and Archimedes method measures the skeletal or true density of a sample. True density is the ratio of the mass of solid material to volume of solid material (not accounting for closed pores). The true volume is measured by gas displacement using Boyle's law, for He pycnometry, or by liquid displacement (buoyancy) using Archimedes method. Helium, or another inert gas, is used as the displacement medium. The true density is calculated by dividing the sample weight by the true volume that is measured by He pycnometry, Archimedes method, or calculated from the densities of the component phases (Rietveld refinement). To determine the porosity the bulk or dry density, i.e., the theoretical density of the sample, calculated from the physical sample dimensions using device(s) like caliper(s), is divided with the true density calculated from the pycnometer or Archimedes measurement, see equation 1 below.

$$Porosity ( \% ) = 1 - ( {P}_{true} / {P}_{theoretical} ) \times 100 \qquad ( eq . 1 )$$

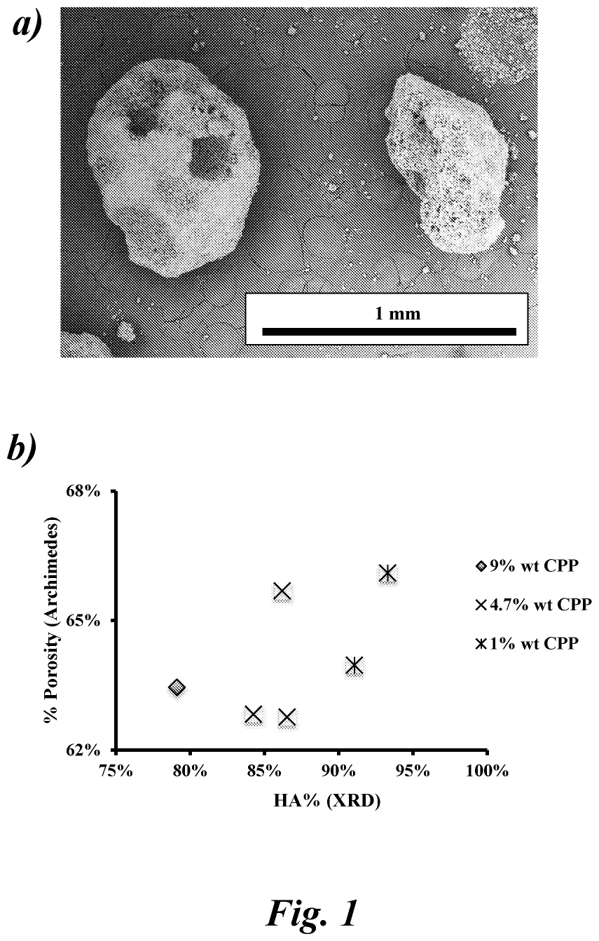

A SEM image of two typical macroporous cement composition particles is shown in FIG. 1a.

The aim of a bone void filler is to function as a template for new bone formation rather than being a permanent bone substitute. In order to do that it is an advantage if the cement composition, i.e., the bone void filler, comprises macropores. Macropores could improve cell colonization within the material and/or increase the osteoclastic degradation.

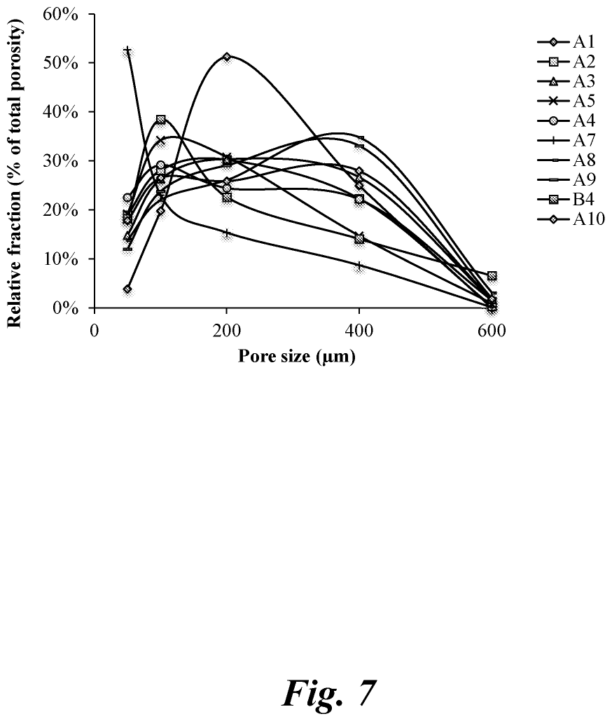

Macropores are defined as pores having a pore diameter >100 μm. In one embodiment of the invention the macropores cement composition has pores with an average pore diameter >100 μm, preferably >150 μm, more preferably >200 μm. The average pore diameter can be determined by, for example, micro-computed tomography (μ-CT), porosimetry, or any other suitable technique which are known to the skilled person. Examples of pore size distributions for compositions according to the invention can be seen in FIG. 7. In FIG. 7, the pore size distribution for the different samples has been determined using microcomputed tomography (μ-CT) for pore size together with Archimedes method for total porosity.

In an aspect of the invention, the porosity of the macroporous cement composition is 60-80 vol %. The porosity can be determined, for example, by helium pycnometry as described above, or by micro-computed tomography (μ-CT), densitometry, or any other suitable technique known to the skilled person. In one embodiment of the invention, the porosity of the macroporous cement composition is 60-75 vol %, such as 60-70 vol %., preferably 62-68 vol %, or more preferably 63-67 vol % as determined by Archimedes method. The graph in FIG. 1b shows the porosity values (in vol %) for four examples of macroporous cement composition according to the invention.

It is an advantage that the macroporous cement composition comprises a majority, e.g., 80 wt % or more, of hydroxyapatite, since it is a well-known phase that is stable, and its in vivo (e.g., rate of degradation and/or resorption) behavior has been studied and is reasonable well understood. It also has advantageous effects on the shelf-life and overall handling properties of the cement. It can be used as a delivery vehicle for other calcium phosphate phases that are beneficial to use in a bone cement.

Hydroxyapatite (HA) as used herein also include various forms of HA including, but not limited to, calcium deficient hydroxyapatite (CDHA), and mixtures of HA and CDHA.

One advantage with the present invention is that a bone cement composition comprising hydroxyapatite and at least one additional calcium phosphate phase (bioactive calcium phosphate phase) is more bioactive than a single-phase cement with only hydroxyapatite. In one embodiment the additional phase is β-calcium pyrophosphate (β-CPP). The additional phase may also be selected from β-calcium pyrophosphate, α-tricalcium phosphate, octacalcium phosphate, and dicalcium phosphate, including any combination thereof.

In one embodiment of the invention, the macroporous cement composition comprises hydroxyapatite, β-calcium pyrophosphate and α-tricalcium phosphate (α-TCP). α-TCP is the calcium salt of phosphoric acid, it has the chemical formula Ca₃(PO₄)₂ and it is a precursor of hydroxyapatite. It is a bioactive material that can be used as a bone replacement to enable the formation of new bones. α-TCP dissolves rapidly in-vivo and releases ions, the rapid release is advantageous in terms of new bone formation.

In one embodiment of the invention, the macroporous cement composition comprises 80-95 wt % hydroxyapatite, Ca₁₀(PO₄)₆(OH)₂, 0.1-10 wt % β-calcium pyrophosphate, Ca₂P₂O₇, preferably 1-10 wt % β-calcium pyrophosphate, and <10 wt % α-tricalcium phosphate, Ca₃(PO₄)₂. Preferably, the macroporous cement composition comprises around 90 wt % hydroxyapatite, Ca₁₀(PO₄)₆(OH)₂, 0.1-10 wt % β-calcium pyrophosphate, Ca₂P₂O₇, preferably 1-10 wt % β-calcium pyrophosphate, and <10 wt % α-tricalcium phosphate, Ca₃(PO₄)₂.

As understood by the skilled person it is possible that a macroporous cement composition additionally comprises minor amounts of impurities such as salts, etc. The amount of impurities is typically <5 wt %, or <3 wt %, or <1 wt %.

B-Calcium pyrophosphate is present as individual particles in the macroporous cement composition. The size of the particles can influence the cell behavior. Too big particles may not react with the cells or may be difficult for cells to degrade, too small particles may dissolve in vivo or be too small to interact with the cells in a beneficial way. It is also possible that the particles effects shelf-life of the composition. In one embodiment of the invention, the average particle size of the β-calcium pyrophosphate is 100 nm-10 μm, or preferably 5-10 μm. The average particle size can be determined by, for example, sieving, scanning electron microscopy (SEM), laser diffraction, electron diffraction, electron or light microscopy, or any other suitable technique known to the skilled person. Using laser diffraction the average particle size is to be regarded as the D₅₀ value or the median particle size value. In a preferred embodiment, the average particle size of the granules is determined by sieving and/or SEM, such as by sieving, by SEM, or by sieving and SEM.

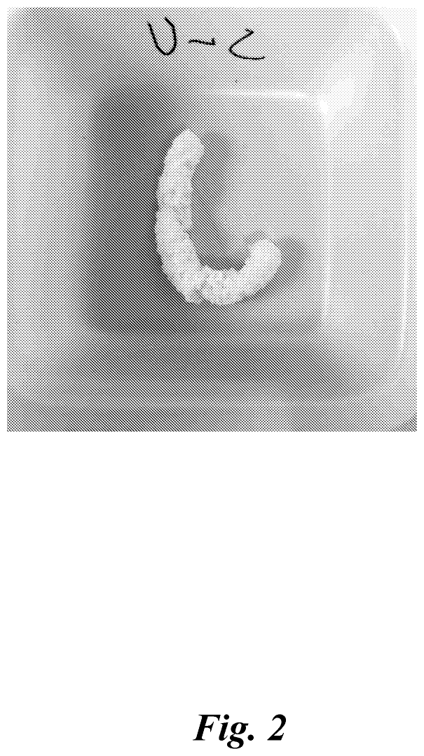

Another factor possibly affecting the performance of a cement composition as a bone void filler is the particle size of the cement composition. The particle size can, for example, affect the injectability in case the cement composition is used as a putty formulation or paste, and the resorption rate of the cement composition. In one embodiment, the average particle size of the composition is 50-800 μm, such as 53-800 μm, or 53-600 μm. The average particle size can be determined by, for example, laser diffraction, scanning electron microscopy, sieving or any other suitable technique as determined by the skilled person. A putty formulation according to the invention, i.e., produced using the macroporous cement composition of the invention, can be seen in FIG. 2.

In an embodiment, about 5 g of granules have the following particle size distribution:
0.46 g of 50-100 μm;
0.91 g of 100-200 μm;
1.36 g of 200-400 μm;
1.14 g of 400-600 μm; and
1.14 g of 600-800 μm.

The macroporous cement composition according to the invention is suitable for use as a bone void filler material. The macroporous cement composition can be formed into granules and mixed with a liquid, such as water, into a putty, paste or similar, after which the putty comprising the granules is injected into a bone void. The putty may optionally comprise a binder, such as carboxymethyl cellulose or poloxamer 407 (P407). Once inserted into a bone void the granules act as a bone replacement and enables the formation of new bone and prevent the formation of fibrous tissue.

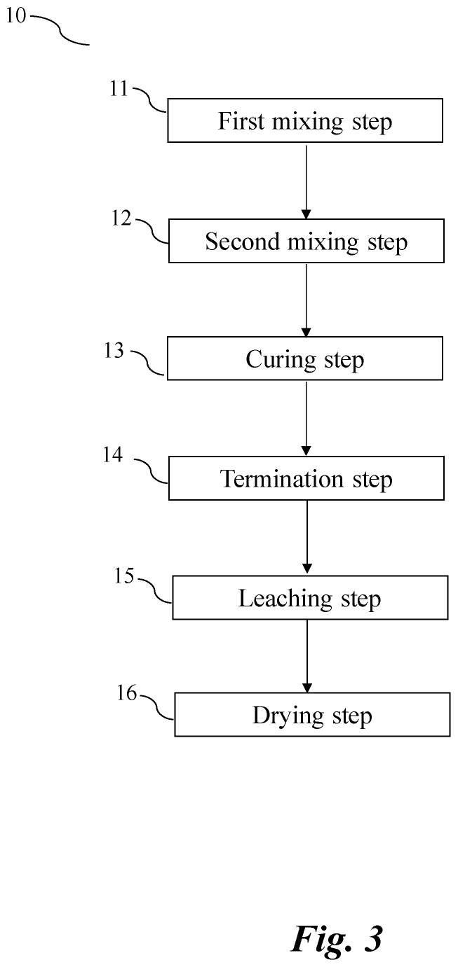

Another aspect of the invention relates to a method 10 for forming a macroporous cement composition, see FIG. 3. The method 10 comprises the steps of:
First mixing step 11: mixing 11 α-tricalcium phosphate (α-TCP), β-calcium pyrophosphate (β-CPP), hydroxyapatite (HA) and a sacrificial phase forming a dry powder mix, wherein all components are in a solid form;
Second mixing step 12: mixing 12 the dry powder mix formed in the first mixing step 11 and a liquid and forming a paste;
Curing step 13: curing 13 the formed paste at a temperature above room temperature, preferably at 50-60° C. for 20-30 hours;
Termination step 14: terminating 14, i.e., halting, the chemical reaction associated with the curing 13 after a predetermined time period selected so that a predetermined amount of α-tricalcium phosphate (α-TCP) remains unreacted forming a solid cement composition, the predetermined amount being above 1 wt %, preferably above 3 wt %;
Leaching step 15: leaching 15 the sacrificial phase solid cement composition; and
Drying step 16: drying 16 at a temperature above room temperature, preferably at 50-60° C. for 20-30 hours, to form the macroporous cement composition.

The method 10 is schematically illustrated in the flowchart in FIG. 3. In the first mixing step 11 solid powders of α-TCP, β-CPP, HA and a sacrificial phase are mixed together. In one embodiment of the invention, the sacrificial phase is polyethylene glycol (PEG), preferably with a molecular weight of approximately 6-35 kDa and/or an average particle size of 100-600 μm. Other examples of sacrificial phases include inorganic salts, e.g., NaCl, MgCl₂, CaCl₂, sugars, polysaccharides, starch, etc. In one embodiment of the invention, the dry powder mix formed in the first mixing step 11 comprises 20-50 wt % PEG, 0.1-10 wt % β-CPP, 0.5-1 wt % HA, and the balance being α-TCP.

The β-CPP used in the method 10 is in solid form, as particles or powder. It is non-soluble in the liquid that is used so it does not dissolve in the second mixing step 12 or in any other of the steps in the method 10.

In the second mixing step 12, the dry powder mix formed in the first mixing step 11 is mixed with a liquid. In one embodiment, the liquid is water, preferably deionized and/or ultrapure water. The ratio of liquid to solid influences the quality of the final macroporous cement composition and is, therefore, of importance. In one embodiment, the amount of liquid is 0.4-0.6 mL/g dry powder mix, i.e., the liquid to powder ratio (L/P) in the second mixing step 12 is 0.4-0.6.

During the curing step 13, the α-TCP is transformed into HA, i.e., the cement sets. However, the curing step 13 may be terminated before all α-TCP has transformed into HA in order for the final composition to comprise some amount α-TCP that is a bioactive phase. However, the amount of α-TCP should not be too high since α-TCP is a reactive material that may transform during storage and, hence, decrease the shelf life of the macroporous cement composition. The reaction could therefore be terminated when about 80 wt % or more of the α-TCP has transformed into HA. The curing time needed for a >80 wt % conversion depend on the scale of the synthesis, the particle size of the powder(s), the temperature, etc. and can be determined by the skilled person using for example powder X-ray diffraction (XRD) and Rietveld analysis. In one embodiment, the curing step 13 is performed at 50-60° C. for 20-30 hours. In the termination step 14, the reaction is terminated. This is, for example, done by submerging the composition obtained from the curing step 13 in a solvent, such as acetone, isopropanol, or ethanol, or by freezing the composition to between −20 to −80° C. for around 4 hours or longer. In such way the conversion of α-TCP to HA is terminated.

The sacrificial phase is preferably not soluble when the dry powder mix is mixed with a liquid in the second mixing step 12. In this way the cured cement is formed around the sacrificial phase so that the sacrificial phase forms an interconnected pore structure in the cured cement. Hence, the sacrificial phase controls the size and connectivity of the formed pore structure. By removing the sacrificial phase after the curing step 13 the sacrificial phase function as a template for the pore structure. In the leaching step 15, the sacrificial phase is removed. In one embodiment, the sacrificial phase is removed by submerging the composition in hot water, e.g., 70-90° C. for 1-24 hours. As appreciated by the skilled person the time required for complete, or almost complete, removal of the sacrificial phase will depend on the type of sacrificial phase, the temperature and type of liquid used for the leaching, the scale of the synthesis, etc. Being aware of such conditions the skilled person can determine suitable conditions using routine experiments.

Optionally, the leaching step 15 may be performed directly after the curing step 13 by first leaching out the sacrificial phase using a solvent e.g., acetone, followed by submerging the composition formed in the curing step 13 in a solvent. In this way the sacrificial phase is removed at the same time as the reaction is halted.

After the leaching step 15 the solid cement composition is dried in a drying step 16. The drying step 16 may optionally be preceded by a washing step, during which the solid cement composition is washed with water or any other suitable solvent(s) in order to remove residuals from the leaching step 15. In the drying step 16 the solid cement composition is dried either at room temperature or at an elevated temperature. Optionally, after the drying step the formed macroporous cement composition may be sterilized for subsequent use.

A macroporous cement composition according to the invention may be used in a putty formulation. In one embodiment of the invention, there is a putty formulation comprising a macroporous cement composition according to the invention.

A putty formulation may comprise a macroporous cement composition in the form of granules mixed with a carrier. The carrier should not react with the macroporous cement composition, it should be water-soluble and biocompatible. The carrier could, for example, comprise water and carboxymethyl cellulose (CMC) and/or a poloxamer, such as poloxamer 407 (P407). In one embodiment, the amount of granules in a putty formulation is 1-50 wt % or 1-40 wt %, preferably 25-50 wt %, such as 30-50 wt % or 40-50 wt %.

Poloxamer is a triblock copolymer comprising, such as consisting of, a central hydrophobic block of polypropylene glycol (PPG) flanked by two hydrophilic blocks of polyethylene glycol (PEG). Poloxamer 407 comprises, on average, two PEG blocks of 101 repeat units and one PPG block of 56 repeat units. Poloxamer 407 is also known as PLURONIC® F-127, KOLLIPHOR® P 407 and SYNPERONIC® PE/F 127.

A putty formulation may be used in order to insert the macroporous cement composition into a bone void, for example by a surgeon. A putty should preferably have good handling properties, such as not being to liquid or to solid, it should be easy to remove from a syringe, and easy to form into a desired shape. Such properties depend on the amount of granules in relation to the amount of carrier, as well as the type of carrier. Optimal mixture of a putty can be determined by the skilled person using routine experiments.

It should be noted that the specific aspects and embodiments described herein may be combined with each other unless explicitly stated otherwise.

### EXAMPLES

A set of samples were prepared according to the method described above and in FIG. 3. In brief, in the first step 1000 mg α-TCP (d₅₀≤6.12 μm, obtained from Innotere) was mixed with 250-1000 mg PEG (100-600 μm), 10 mg HA seed crystals (particle size <0.063 μm) and 10-140 mg β-CPP. Two types of β-CPP were used, one coarse (d₅₀=8.24 μm) and one fine (d₅₀=1.55 μm). In the second step, the powder obtained in the first step was mixed with deionized water using a L/P of 0.4-0.6. The composition was cured at 50-60° C. for 20-30 hours, after which it was submerged in ethanol in the termination step. The PEG (the sacrificial phase) was removed by submerging the composition in water (70-90° C.) for ˜24 hours. Finally, the composition was dried at 40° C. for 24 hours.

A summary of the formed samples is shown in Table 1 below.

TABLE 1
Summary of prepared samples

| Sample | α-TCP | PEG (1- | β-CPP | β-CPP | | | |
|---|---|---|---|---|---|---|---|
| name | [mg] | 600 μm) [mg] | (coarse) [mg] | (fine) [mg] | HA [mg] | Liquid | L/P [μL] |
| A1 | 1000 | 250 | 140 | 10 | 10 | Water | 600 |
| A2 | 1000 | 250 | 90 | 10 | 10 | Water | 600 |
| A3 | 1000 | 250 | 40 | 10 | 10 | Water | 600 |
| A4 | 1000 | 250 | 0 | 10 | 10 | Water | 600 |
| A5 | 1000 | 250 | 0 | 50 | 10 | Water | 600 |
| A6 | 1000 | 250 | 0 | 100 | 10 | Water | 600 |
| A7 | 1000 | 250 | 50 | 0 | 10 | Water | 600 |
| A8 | 1000 | 250 | 100 | 0 | 10 | Water | 600 |
| A9 | 1000 | 250 | 10 | 10 | 10 | Water | 600 |
| A10 | 1000 | 250 | 25 | 25 | 10 | Water | 600 |
| A11 | 1000 | 250 | 50 | 50 | 10 | Water | 600 |
| B1 | 1000 | 1000 | 0 | 10 | 10 | Water | 400 |
| B2 | 1000 | 1000 | 0 | 10 | 10 | Water | 600 |
| B3 | 1000 | 0 | 0 | 10 | 10 | Water | 400 |
| B4 | 1000 | 0 | 0 | 10 | 10 | Water | 600 |
| B5 | 1000 | 250 | 0 | 0 | 10 | Water | 600 |

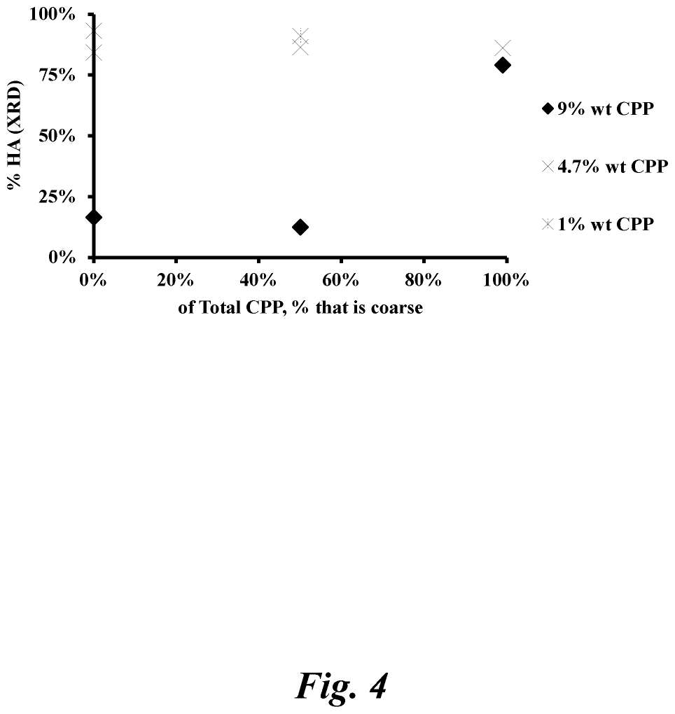

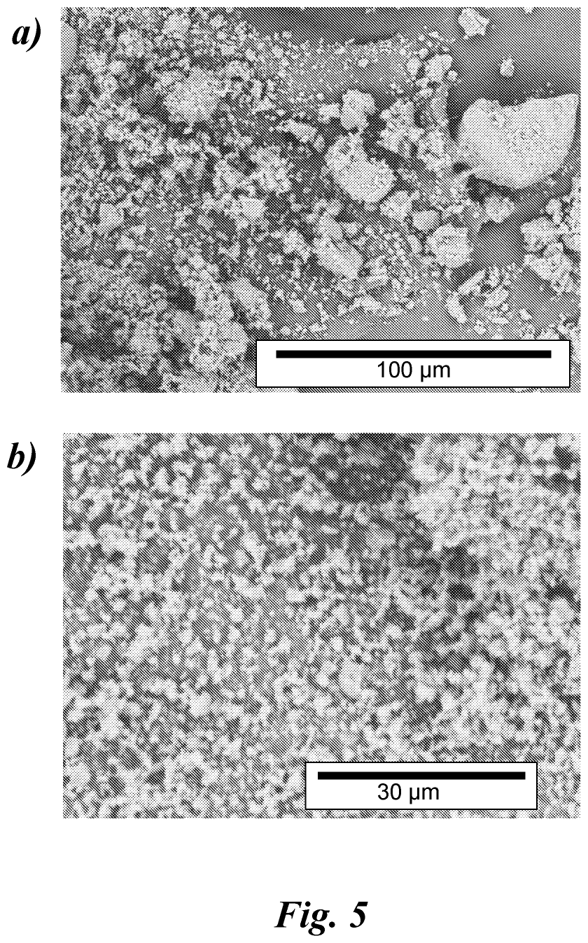

Sample A6 and A11 comprising 100% and 50% fine β-CPP, respectively did not set and were, hence, not analyzed further. The graph in FIG. 4 shows this, that when the amount of coarse β-CPP is too little almost no HA is formed, i.e. the sample does not set. Hence, large amounts of fine β-CPP inhibits the HA reaction. SEM images of coarse respectively fine β-CPP particles can be seen in FIG. 5, FIG. 5a shows an image of coarse particles and FIG. 5b shows an image of fine particles.

The B-samples (B1-B5) all comprised a larger amount of PEG and a lesser amount of β-CPP. Samples containing more than 40% PEG did not set, consistently. The A1, A6 and B2 samples did not set and were, hence, not analyzed further.

The remaining samples were analyzed for porosity and composition. The composition was analyzed using XRD and Rietveld refinement. The porosity was analyzed using Archimedes principle wherein wet density is compared to dry density, He pycnometry, XRD and μCT.

The results from the analyzed samples are summarized in Table 2 below, and in FIG. 1b that is a graph showing amount of HA vs Archimedes porosity.

TABLE 2
Summary of results from composition and porosity analysis.

| | | | Archimedes | Pycnometer | XRD |
|---|---|---|---|---|---|
| Sample | β-CPP | HA (formed) | porosity | porosity | porosity |
| name | [wt %] | [wt %] | [vol %] | [vol %] | [vol %] |
| A2 | 9.0 | 83.0 | 62.3 | — | — |
| A3 | 4.7 | 86.9 | 63.3 | — | — |
| A4 | 1.0 | 93.3 | 66.1 | 68.7 | 72.0 |
| A5 | 4.7 | 84.3 | 62.8 | 67.2 | 70.3 |
| A7 | 4.7 | 86.2 | 65.7 | 67.8 | 70.9 |
| A8 | 9.0 | 79.1 | 63.5 | 66.6 | 69.7 |
| A9 | 1.0 | 91.1 | 64.0 | 68.4 | 71.6 |
| A10 | 4.7 | 86.5 | 62.8 | 67 | 70.2 |
| B1 | | | | | |
| B3 | | | | | |
| B4 | 1.0 | 94 | 58.8 | — | — |
| B5 | | | | | |

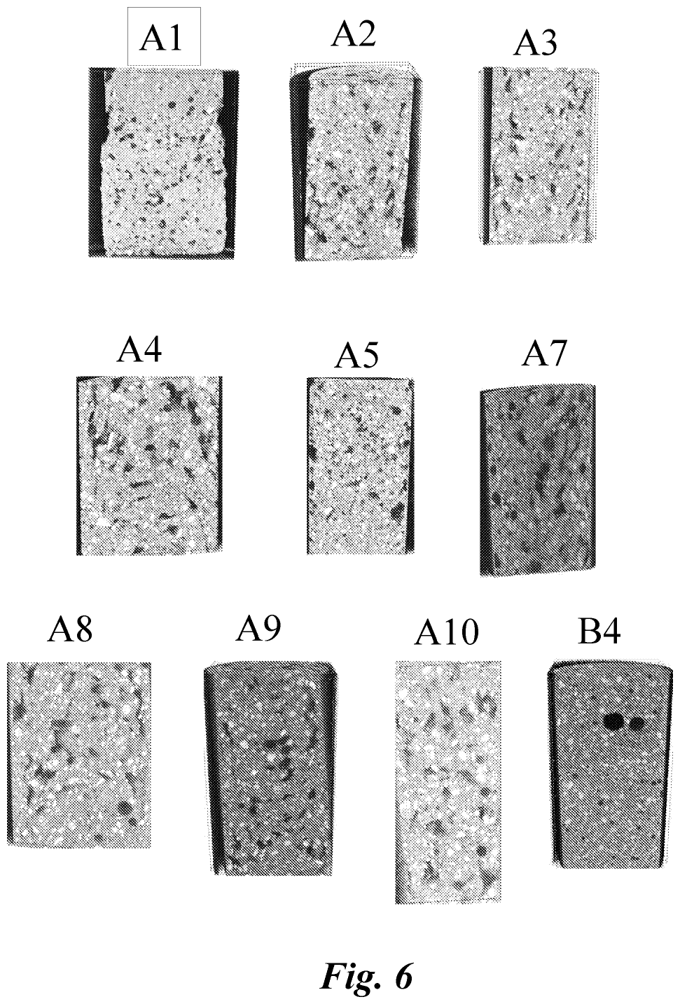

The samples were further analyzed using a μCT in order to visualize the porous structure and determine the pore size distribution (same method as in WO 2015/162597). The results can be seen in FIGS. 6 and 7. FIG. 6 shows the images, as can be seen in the figure all analyzed samples have visible pores. FIG. 7 shows the pore size distributions for the different samples, as can be seen all samples has pores with pore diameters between 50-600 μm and an average pore size around 250 μm.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A macroporous cement composition comprising:
80-95 wt % hydroxyapatite; and,
β-calcium pyrophosphate, wherein the porosity of the macroporous cement composition is 60-80 vol % as determined using Archimedes method.

2. The macroporous cement composition according to claim 1, further comprising α-tricalcium phosphate.

3. The macroporous cement according to claim 2, wherein the macroporous cement composition comprises:
80-95 wt % hydroxyapatite;
0.1-10 wt % β-calcium pyrophosphate, preferably 1-10 wt % β-calcium pyrophosphate; and
<10 wt % α-tricalcium phosphate.

4. The macroporous cement according to claim 3, wherein the macroporous cement composition comprises:
around 90 wt % hydroxyapatite;
0.1-10 wt % β-calcium pyrophosphate, 1-10 wt % β-calcium pyrophosphate; and
<10 wt % α-tricalcium phosphate.

5. The macroporous cement according to claim 1, wherein 25-75% of the total pore volume as determined by Archimedes method is constituted by pores having a pore diameter of 100-600 μm as determined by micro-computed tomography.

6. The macroporous cement composition according to claim 1, wherein the mean particle size of the β-calcium pyrophosphate in the macroporous cement composition is 500 nm-10 μm as determined by sieving and/or scanning electron microscopy.

7. The macroporous cement composition according to claim 1, wherein the average particle size of the macroporous cement composition is 50-800 μm as determined by laser diffraction.

8. A method of manufacturing a macroporous cement composition, wherein the method comprises the steps of:
mixing α-tricalcium phosphate, β-calcium pyrophosphate, and a sacrificial phase forming a dry powder mix, wherein all components are in solid form;
mixing the dry powder mix and a liquid forming a paste;
curing the paste at a temperature above room temperature;
terminating the chemical reaction associated with the curing after a predetermined time period selected so that a predetermined amount of α-tricalcium phosphate remains unreacted forming a solid cement composition, the predetermined amount being above 1 wt %
leaching the sacrificial phase from the solid cement composition; and
drying at a temperature above room temperature to form the macroporous cement composition.

9. The method according to claim 8, wherein the average particle size of the β-calcium pyrophosphate particles is 500 nm-10 μm as determined by laser diffraction.

10. The method according to claim 8, wherein the sacrificial phase is polyethylene glycol.

11. The method according to claim 8, wherein
the liquid is water; and
mixing the dry powder mix comprises mixing the dry powder mix with 0.4-0.6 mL water/g of the dry powder mix.

12. The method according to claim 8, wherein terminating the chemical reaction comprises submerging the composition formed in the curing step in a solvent.

13. The method according to claim 8, wherein the manufactured macroporous cement composition comprises:
80-95 wt % hydroxyapatite; and,
β-calcium pyrophosphate, wherein the porosity of the macroporous cement composition is 60-80 vol % as determined using Archimedes method.

14. A putty formulation comprising the macroporous cement composition according to claim 1.

15. The putty formulation according to claim 14, further comprising a carrier.

16. The method according to claim 8, wherein curing the paste comprises curing the paste at 50-60° C. for 20-30 hours.

17. The method according to claim 8, wherein the predetermined amount is above 3 wt %.

18. The method according to claim 8, wherein drying comprises drying at 50-60° C. for 20-30 hours to form the macroporous cement composition.
